(19) United States
(12) Patent Application Publication
(10) Pub. No.: US 2026/0102477 A1
(43) Pub. Date: Apr. 16, 2026

(54) Tumor-Associated Antigen Based Cancer Vaccine

(71) Applicant: South Dakota Board of Regents, Pierre, SD (US)

(72) Inventors: Wenfeng An, Pierre, SD (US); Raj Nandani, Jacksonville, FL (US)

(21) Appl. No.: 19/359,820

(22) Filed: Oct. 16, 2025

Related U.S. Application Data
(60) Provisional application No. 63/708,075, filed on Oct. 16, 2024.

(51) Int. Cl. A61K 39/00; A61K 9/00; A61K 31/7105; A61K 31/7115; A61K 45/06; A61P 35/00

(52) U.S. Cl. CPC A61K 39/001102; A61K 9/0019; A61K 31/7105; A61K 31/7115; A61K 45/06; A61P 35/00; A61K 2039/53; A61K 2039/54; A61K 2039/55555; A61K 2039/55588; A61K 2039/572; A61K 2039/6018

(57) ABSTRACT

Disclosed herein is a composition comprising a nucleic acid encoding long interspersed elements type 1 open reading frame 1 protein (LINE-1 ORF1p) or a functional variant thereof, and a nanoparticle. Also disclosed are methods of using the composition to elicit an immune response in methods of treating subjects, such as those with cancer.

### PRIORITY STATEMENT

This application claims benefit of U.S. Provisional Application No. 63/708,075, filed Oct. 16, 2024, incorporated herein by reference in its entirety.

### GOVERNMENT SUPPORT CLAUSE

This invention was made with government support under GM145398 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Oct. 15, 2025, is entitled “11214-014US1_ST26.xml”, and is 4,692 bytes in size.

### BACKGROUND

Since the early 1990s, nucleic acids such as DNA and RNA have emerged as potent tools for gene-based therapeutics and vaccine development. The groundbreaking discovery by Acsadi and colleagues in 1990 demonstrated that injecting nucleic acids could lead to protein expression in host cells, thereby stimulating immune responses (Acsadi et al., 1991). This initiated a new era of nucleic acid-based vaccine development, diverging from traditional approaches that relied on attenuated or inactivated pathogens such as those used for polio, smallpox, and measles (Geall et al., 2013; Wang et al., 1997).

As the field advanced, mRNA emerged as an up-and-coming alternative to DNA vaccines due to its cytoplasmic activity, absence of genomic integration risk, and ease of manufacturing. Unlike DNA, mRNA does not require nuclear entry, and its translation occurs directly in the cytoplasm, providing a safer and more efficient platform for transient antigen expression (Fuller & Berglund, 2020; Karikó et al., 2008).

Despite early skepticism around mRNA stability, a series of modifications and technological advancements have transformed the field. Notably, pseudouridine modifications and codon optimization increased mRNA translation efficiency and reduced innate immune activation (Pardi et al., 2018b). Furthermore, lipid nanoparticles (LNPs) emerged as a robust delivery system, protecting mRNA from degradation and facilitating targeted intracellular delivery (Islam et al., 2015).

Lipid nanoparticles (LNPs) are currently the most efficient and widely used delivery systems for mRNA vaccines. LNPs encapsulate the negatively charged and fragile mRNA molecules, shielding them from enzymatic degradation by extracellular RNases and enabling their efficient transport into the cytoplasm. Their structural components, including ionizable lipids, cholesterol, phospholipids, and polyethylene glycol (PEG) lipids, help in enhancing stability, facilitating endosomal escape, and improving cellular uptake. LNPs also offer advantages of scalability, biocompatibility, and tunable biodistribution.

In addition to infectious diseases, the last decade has witnessed a significant pivot towards mRNA vaccines for cancer therapy. Cancer vaccines aim to stimulate the immune system to target tumor-associated antigens (TAAs) or tumor-specific neoantigens that allow tumors to evade immune detection. mRNA is particularly advantageous in oncology because it allows for rapid, flexible design to encode personalized neoantigens and multiple epitopes in a single construct (Sahin & Türeci, 2018).

Recent studies have demonstrated the therapeutic efficacy of mRNA vaccines in clinical settings. A personalized mRNA vaccine (mRNA-4157/V940) targeting melanoma neoantigens, when combined with immune checkpoint blockade (pembrolizumab), resulted in a 44% reduction in recurrence or death (NCT03897881). Another example includes autogene cevumeran, an individualized mRNA vaccine targeting pancreatic cancer neoantigens, which showed strong immune responses and improved recurrence-free survival (Rojas et al. 2023).

What is needed in the art is the identification of an effective, broad acting TAA target.

### SUMMARY

The present invention relates to nucleic acid-based compositions and methods for eliciting an immune response against tumors expressing the Long Interspersed Element-1 (LINE-1) open reading frame 1 protein (ORF1p). In certain embodiments, the composition comprises a nucleic acid molecule encoding LINE-1 ORF1p or a functional variant thereof, formulated within a nanoparticle delivery system. The nucleic acid may be messenger RNA (mRNA) that is codon-optimized for mammalian expression, includes modified nucleosides such as N1-methylpseudouridine, is flanked by 5′ and 3′ untranslated regions, and contains a co-transcriptional cap structure.

In some embodiments, the nanoparticle component is a lipid nanoparticle (LNP) composed of ionizable lipids, cholesterol, phospholipids, and polyethylene glycol (PEG)-lipids. Alternative embodiments utilize polymeric nanoparticles, optionally containing targeting ligands, adjuvants, DSPC, or cryoprotectants to enhance delivery, immune stimulation, and formulation stability. The compositions are designed for delivery to antigen-presenting cells to induce a cytotoxic T-lymphocyte response against ORF1p-expressing tumor cells.

The invention further provides methods for eliciting an immune response in a subject by administering the nanoparticle-encapsulated nucleic acid composition. In some embodiments, the subject has a tumor characterized by LINE-1 ORF1p overexpression. The composition can be formulated as a therapeutic vaccine and may be co-administered with other anti-cancer agents or adjuvants. Additional embodiments include nanoparticles engineered for enhanced endosomal escape and targeted delivery to tumor or antigen-presenting cells through ligand conjugation. These methods and compositions collectively enable targeted immunotherapy against multiple tumor types that express LINE-1 ORF1p.

### BRIEF DESCRIPTION OF THE FIGURES

FIG. 1 shows the mechanism of action for mRNA cancer vaccines. mRNA vaccine can induce both cellular and humoral immunity against cancer cells. (i.m., intramuscular; s.c., subcutaneous; ADCC, antibody-dependent cell-mediated cytotoxicity by NK cells.)

FIG. 2A-E shows the process of synthesizing ORF1p mRNA. A) sequencing components; B) constructing a DNA template; C) in vitro transcription; D) integrity of mRNA; E) functional evaluation in cells.

FIG. 3A-E shows formulating mRNA into a lipid nanoparticle. A) workflow; B) integrity of mRNA in LNP; C) mRNA-LP size and PDI; D) zeta potential; E) encapsulation efficiency.

FIG. 4A-D shows the frequency of activated splenic T cells in mice vaccinated with PBS saline or ORF1p mRNA LNP. A) Frequency of IFN-γ⁺ CD8⁺ T cells; B) Frequency of TNF-α⁺ CD8⁺ T cells; C) Frequency of IFN-γ⁺ CD4⁺ T cells; D) Frequency of TNF-α CD4⁺ T cells. Splenocytes were quantified under stimulated and unstimulated conditions for surface markers and intracellular targets by flow cytometry (n=5). The statistical test was a one-way ANOVA, and the ** p<0.01.

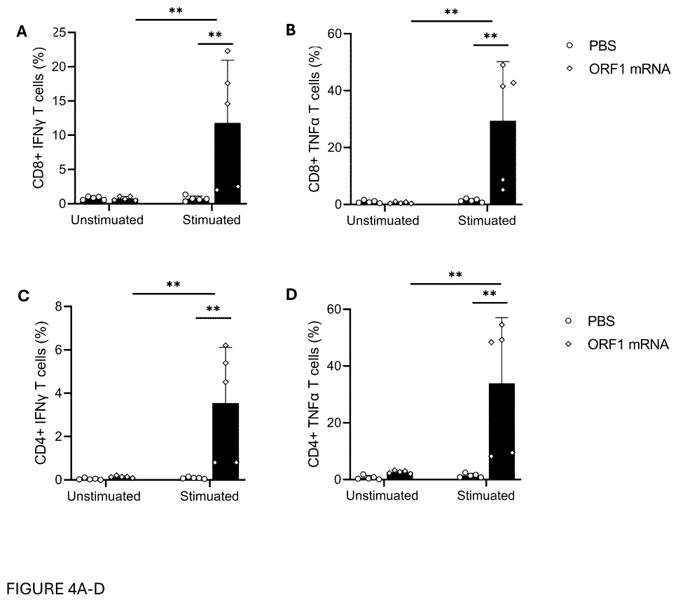

FIG. 5A-B shows the creation of syngeneic mouse cancer cell lines expressing mouse LINE-1 ORF1p.

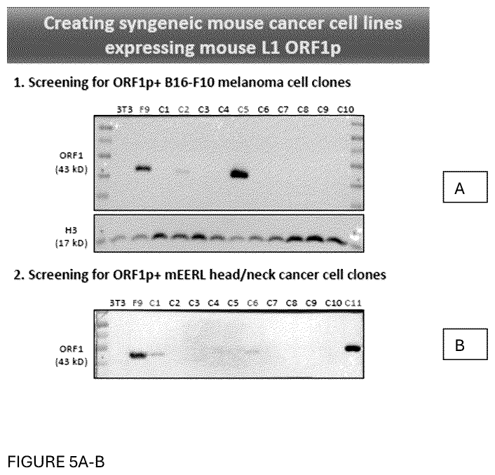

### DEFINITIONS

The following definitions are provided to facilitate understanding of certain terms used throughout this disclosure.

“LINE-1 ORF1” or “ORF1p” refers to the open reading frame 1 protein encoded by long interspersed element-1 (LINE-1 or L1) retrotransposons, which are autonomous human retroelements. ORF1p is an RNA-binding protein involved in retrotransposition and is aberrantly expressed in many human cancers. As used herein, the term encompasses the full-length native protein as well as any functional fragments, homologs, or variants that retain one or more antigenic or biological properties of the native protein, such as immunogenicity or RNA-binding activity.

“Functional variant” refers to any modified form of a nucleic acid or protein that retains at least one functional property of the reference molecule. For ORF1p, such function may include antigenicity, the ability to elicit a T cell response, or retention of epitopes recognizable by antibodies or T cell receptors. Functional variants may include conservative amino acid substitutions, deletions, insertions, or fusion constructs with other domains.

“Nucleic acid” refers broadly to any polynucleotide molecule, including DNA, RNA, cDNA, mRNA, or synthetic analogs thereof, whether naturally occurring or chemically modified. The term encompasses codon-optimized sequences, modified nucleotides such as pseudouridine or N1-methylpseudouridine, and sequences containing untranslated regions (UTRs), poly (A) tails, or cap structures.

“mRNA” refers to messenger RNA capable of being translated into a protein in a host cell. The term includes in vitro transcribed (IVT) mRNA containing modified nucleotides to enhance stability and translational efficiency. Such mRNA molecules may be capped, polyadenylated, and flanked by 5′ and 3′ untranslated regions optimized for expression in mammalian cells.

“Nanoparticle” refers to a particulate carrier system typically ranging in size from about 10 nanometers to about 1000 nanometers, which is capable of encapsulating, protecting, or delivering nucleic acids or other therapeutic molecules to target cells. Nanoparticles include lipid nanoparticles (LNPs), polymeric nanoparticles, micelles, and liposomes, and may contain ionizable lipids, cholesterol, phospholipids, and polyethylene glycol (PEG)-lipids.

“Lipid nanoparticle (LNP)” refers to a nanoparticle composed primarily of lipids that facilitate the encapsulation and delivery of nucleic acids. LNPs generally comprise an ionizable lipid, a phospholipid such as DSPC (1,2-distearoyl-sn-glycero-3-phosphocholine), cholesterol, and a PEG-lipid component. These particles may further contain targeting ligands, adjuvants, or cryoprotectants to enhance delivery efficiency, stability, or immunogenicity.

“Targeting ligand” refers to any molecule, including antibodies, peptides, aptamers, carbohydrates, or small molecules, that selectively binds to a target cell surface marker to direct nanoparticle delivery. Suitable targeting ligands include, for example, folate, RGD peptides, antibodies against PD-L1 or EGFR, or mannose moieties to target dendritic cells.

“Adjuvant” refers to any substance that enhances, potentiates, or modulates an immune response to an antigen. Adjuvants include toll-like receptor (TLR) agonists, cytokines, lipid A analogs, saponins, alum, or STING agonists. The term includes adjuvants incorporated into nanoparticle formulations or co-administered separately.

“Cryoprotectant” refers to a stabilizing compound that prevents nanoparticle degradation or aggregation during freezing, lyophilization, or storage. Examples include sucrose, trehalose, mannitol, and glycine.

“Antigen-presenting cell (APC)” refers to a cell capable of processing and presenting antigens to T cells via major histocompatibility complex (MHC) molecules. APCs include dendritic cells, macrophages, and B cells, and are essential for initiating adaptive immune responses.

“Immune response” refers to the activation or modulation of the immune system, including humoral, cellular, and innate components. An antigen-specific immune response includes activation of cytotoxic T lymphocytes (CTLs), helper T cells, or B cells that recognize an antigen such as ORF1p.

“Cytotoxic T lymphocyte (CTL) response” refers to the activation and expansion of CD8″ T cells that recognize antigenic peptides presented by MHC class I molecules, leading to the lysis of target cells or the secretion of effector cytokines such as interferon-gamma (IFN-γ).

“Vaccine” refers to a pharmaceutical composition designed to induce an immune response that provides prophylactic or therapeutic protection against disease. In certain embodiments, the vaccine is an mRNA-LNP formulation encoding an antigen such as ORF1p, which elicits antigen-specific T cell responses targeting tumor cells that express the encoded antigen.

“Nanoparticle vaccine” refers to any vaccine formulation in which the antigen, nucleic acid, or immunostimulatory agent is encapsulated within, attached to, or complexed with nanoparticles. Nanoparticle vaccines enhance antigen stability, protect nucleic acids from degradation, and promote delivery to antigen-presenting cells, resulting in potent cellular and humoral immune responses. Such vaccines may use lipid, polymeric, or hybrid nanoparticles and can include adjuvants, targeting ligands, or ionizable components to improve efficacy.

“mRNA vaccine” refers to a composition comprising a messenger RNA encoding a target antigen formulated in a delivery system such as a lipid nanoparticle. Upon administration, the mRNA is taken up by host cells, translated into protein, and presented to the immune system to induce antigen-specific immunity. The mRNA vaccine may include modified nucleotides, UTRs, poly (A) tails, and cap structures that enhance expression and stability.

“Therapeutically effective amount” refers to an amount of the composition sufficient to produce a measurable biological or clinical effect, such as an immune response, reduction of tumor burden, or inhibition of disease progression.

“Subject” or “Patient” refers to any mammal, preferably a human, who receives the composition described herein for preventive, therapeutic, or diagnostic purposes.

“Overexpression” refers to the elevated expression of a gene or protein relative to normal tissue levels. In the present disclosure, overexpression of LINE-1 ORF1p is characteristic of certain malignancies and provides a tumor-associated antigen for immunotherapeutic targeting.

“Endosomal escape” refers to the process by which a nanoparticle or its cargo exits the endosome after cellular uptake, allowing release of the nucleic acid into the cytoplasm for translation. LNPs may be engineered with ionizable lipids or fusogenic components to enhance endosomal escape.

“Co-administration” refers to the administration of two or more therapeutic agents either simultaneously or sequentially to achieve a synergistic or enhanced therapeutic effect. Examples include co-administration of an ORF1p mRNA-LNP vaccine with an immune checkpoint inhibitor, chemotherapeutic, or radiotherapy.

“Synergistic immune response” refers to an immune effect that exceeds the additive responses produced by individual agents administered separately. Such synergy may be reflected by enhanced CTL activation, cytokine production, or tumor regression.

“Tumor” or “Cancer” refers to any malignant or neoplastic growth characterized by uncontrolled proliferation of abnormal cells. Tumors may include head and neck cancers, breast cancers, lung cancers, ovarian cancers, pancreatic cancers, or colorectal carcinomas that overexpress LINE-1 ORF1p.

“Encapsulation” refers to the process of incorporating a nucleic acid within or onto a nanoparticle matrix, thereby protecting the cargo from degradation and enabling efficient delivery to target cells.

“Transfection” or “Delivery” refers to the introduction of nucleic acids into a cell using a carrier such as a lipid or polymeric nanoparticle, resulting in expression of the encoded protein.

“Pharmaceutically acceptable carrier” refers to a physiologically compatible medium used to formulate the composition for administration, including buffers, stabilizers, saline, lipids, or polymers that are non-toxic to the subject.

“Combination therapy” refers to a therapeutic strategy involving administration of the disclosed composition in conjunction with one or more additional therapies, including checkpoint inhibitors, chemotherapeutic agents, radiotherapy, or adjuvant immunotherapies.

“Humanized mouse model” refers to an immunodeficient mouse engrafted with human hematopoietic or immune cells, allowing assessment of human immune responses in vivo following vaccination or therapy.

“Regulated translation” refers to the controlled synthesis of a protein mediated by specific nucleotide sequences within untranslated regions or by structural elements that influence mRNA stability, localization, or translational efficiency.

“Codon optimization” refers to the modification of a nucleotide sequence to improve translational efficiency in a target organism by employing codons preferred by the host's tRNA pool. Codon-optimized mRNAs typically yield higher levels of protein expression in mammalian cells.

“Formulated for delivery to antigen-presenting cells” refers to a composition designed by manipulation of particle size, surface charge, lipid composition, or targeting ligands to promote preferential uptake by dendritic cells or macrophages, thereby enhancing immune activation.

“Nanoparticle-based immunotherapy” refers to a therapeutic strategy using nanoparticles as delivery systems to induce or modulate immune responses against disease. Such systems may deliver mRNA, peptides, or proteins encoding tumor antigens to stimulate antigen-specific T cell activation and tumor clearance.

“Self-assembling lipid nanoparticle” refers to a particle that spontaneously forms upon mixing an aqueous nucleic acid solution with an ethanol lipid mixture, resulting in encapsulation of the nucleic acid within a lipid matrix without the need for surfactants or high-shear mixing.

“Stabilized mRNA formulation” refers to an mRNA composition protected against degradation by inclusion of modified nucleotides, optimized UTRs, and formulation in a lipid or polymeric nanoparticle with excipients such as cryoprotectants and antioxidants.

### DETAILED DESCRIPTION

The present invention is based on the finding that an mRNA vaccine targeting LINE-1 ORF1 protein (ORF1p) is immunogenic in wild-type mice. LINE-1 (long interspersed element type 1) represents a class of abundant repetitive sequences that can replicate itself in mammalian genomes via a copy-and-paste mechanism. LINE-1 ORF1p is only expressed in cancer cells but is undetectable in normal somatic cells. An added advantage is that ORF1p is present in nearly half of the cancer samples across 25 cancer histotypes examined (Rodic et al., 2014). Therefore, a LINE-1 ORF1p-based cancer vaccine is broadly applicable to many patients with a diverse range of cancer types.

### Compositions

In one aspect, the invention provides a composition comprising a nucleic acid encoding LINE-1 ORF 1p or a functional variant thereof, and a nanoparticle suitable for in vivo delivery. The composition may further comprise one or more excipients, adjuvants, or stabilizers. The nucleic acid may be a DNA, RNA, or synthetic analog capable of expressing the ORF1p polypeptide in mammalian cells. In preferred embodiments, the nucleic acid is messenger RNA (mRNA). The mRNA may encode the full-length LINE-1 ORF1 protein or an immunogenic fragment thereof that includes one or more epitopes recognized by cytotoxic T lymphocytes (CTLs).

The mRNA may be codon-optimized for mammalian expression, employing codon bias preferred in human or murine cells. In some embodiments, optimization may increase GC content or eliminate inhibitory sequences such as cryptic splice sites or RNA instability motifs. The mRNA may include regulatory elements such as Kozak sequences, enhancers, or stabilizing motifs. Exemplary optimized sequences may increase translation efficiency by at least about 25%, 50%, or 100% relative to native coding sequences, for example.

In certain embodiments, the mRNA comprises modified nucleotides designed to enhance stability and translation while reducing innate immune recognition. Suitable modifications include, without limitation, N1-methylpseudouridine, 5-methylcytidine, pseudouridine, and 2-thiouridine. The ratio of modified to unmodified nucleotides may range from about 10% to about 100%. In some embodiments, all uridines are substituted with N1-methylpseudouridine to improve expression and reduce interferon activation.

The mRNA may further include untranslated regions (UTRs) flanking the coding sequence. The 5′ UTR may be derived from mammalian globin genes, such as alpha- or beta-globin, or from viral sequences known to enhance translation. The 3′ UTR may be derived from human growth hormone, beta-globin, or synthetic stabilizing elements. The UTRs may together improve mRNA stability and translational efficiency by at least about 2-fold. The mRNA may further comprise a poly (A) tail of about 80 to about 200 nucleotides in length, preferably about 120 to about 150 nucleotides, for example.

The mRNA can be capped with a co-transcriptional or enzymatic cap structure to facilitate efficient ribosomal loading. Exemplary caps include CleanCap, ARCA, or m7GpppG cap analogs. The capped mRNA may be formulated in aqueous buffer at concentrations ranging from about 0.1 to about 5 mg/mL prior to encapsulation.

The composition may also contain untranslated sequences designed to regulate translation or localization. For example, UTRs may include microRNA-binding sites or stem-loop structures conferring tissue-specific expression. In one embodiment, translation occurs predominantly in antigen-presenting cells, enhancing immune priming while minimizing off-target expression.

### Nanoparticle Delivery Systems

In another aspect, the invention provides nanoparticles capable of encapsulating or complexing the mRNA described herein. The nanoparticles protect the mRNA from degradation and promote uptake into target cells, such as dendritic cells or tumor cells. The nanoparticles may be lipid-based, polymeric, or hybrid in nature.

In some embodiments, the nanoparticles are lipid nanoparticles (LNPs). LNPs typically comprise four main components: (i) an ionizable lipid, (ii) a phospholipid, (iii) cholesterol, and (iv) a PEG-lipid. The ionizable lipid facilitates endosomal escape by becoming positively charged at low pH. Suitable ionizable lipids include DLin-MC3-DMA, ALC-0315, SM-102, DODMA, or proprietary analogs having tertiary amine groups. The phospholipid, such as DSPC or DOPE, provides membrane stability and structure. Cholesterol enhances rigidity and fusion capacity, while PEG-lipids such as PEG-DMG control particle size and circulation time.

The molar ratios of these components may vary. In one embodiment, the LNP comprises about 40-60 mol % ionizable lipid, about 10-20 mol % phospholipid, about 30-40 mol % cholesterol, and about 1-5 mol % PEG-lipid. In another embodiment, the ratio of ionizable lipid to mRNA is from about 3:1 to about 10:1 (w/w). The resulting LNPs typically have diameters of about 50 to about 200 nm, with polydispersity indices less than 0.2.

In alternative embodiments, the nanoparticles are polymeric, comprising biodegradable or biocompatible polymers such as PLGA, PBAE, chitosan, or polyethyleneimine. The polymeric nanoparticles may encapsulate mRNA via electrostatic complexation or emulsion methods. Polymer:mRNA ratios may range from about 1:1 to about 50:1 (w/w). Polymeric nanoparticles may further be coated with lipids or PEG to enhance biocompatibility.

In yet other embodiments, hybrid nanoparticles combining lipid and polymer components are employed. For example, an mRNA-loaded PLGA core may be coated with a lipid monolayer containing DSPC and cholesterol, creating a lipopolyplex structure that combines stability with efficient delivery.

Nanoparticles may include additional agents such as targeting ligands. Targeting ligands facilitate binding to specific cells or tissues, enhancing delivery precision. Suitable ligands include antibodies, antibody fragments (Fab, scFv), aptamers, peptides, or small molecules. Examples include mannose (for dendritic cell targeting), folate (for ovarian tumors), RGD peptides (for integrin-expressing cells), or transferrin (for brain delivery).

The nanoparticle formulation may further contain adjuvants to boost immune activation. Exemplary adjuvants include TLR agonists (e.g., CpG, poly (I:C), R848), STING agonists, or immunostimulatory lipids such as monophosphoryl lipid A. Adjuvant concentrations may range from about 0.001% to about 2% (w/v) of the total formulation.

The composition may also include cryoprotectants and stabilizers, such as trehalose, sucrose, mannitol, or glycine, which prevent aggregation and preserve particle integrity during freezing or lyophilization. In certain embodiments, the formulation remains stable for at least one month at 4° C. or six months at −20° C. with no more than 10% degradation of encapsulated mRNA.

### Mechanism of Action

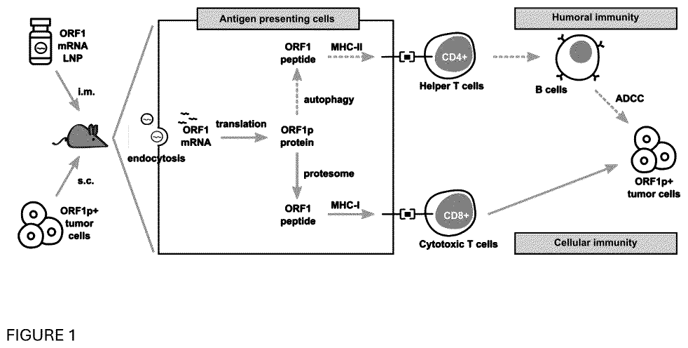

Upon administration, the mRNA-LNP composition is taken up by host cells via endocytosis. The ionizable lipid component promotes endosomal escape, releasing the mRNA into the cytoplasm. The host translational machinery then synthesizes the ORF1p protein, which undergoes intracellular processing to yield antigenic peptides. These peptides are presented by MHC class I and II molecules, thereby activating both CD8 cytotoxic T lymphocytes (CTLs) and CD4 helper T cells (FIG. 1).

The resulting immune response is antigen-specific, leading to selective targeting of tumor cells expressing endogenous ORF1p. Activated CTLs secrete perforin, granzyme B, and interferon-gamma, inducing apoptosis of ORF1p-positive cancer cells. Helper T cells and dendritic cells amplify this response, promoting long-term immunological memory.

In certain embodiments, the immune response may be further enhanced by co-administration of immunomodulatory agents, such as checkpoint inhibitors (anti-PD-1, anti-PD-L1, anti-CTLA-4), cytokines (IL-2, IL-12, GM-CSF), or costimulatory molecules (OX40L agonists, CD40 antibodies). These agents may be administered concurrently, sequentially, or incorporated directly into the nanoparticle formulation.

The mechanism of action further includes stimulation of innate immunity via pattern recognition receptors. In vitro transcribed unmodified RNA may activate TLR3, TLR7, or TLR8 pathways, leading to secretion of type I interferons and further immune amplification.

### Methods of Use

In another aspect, the invention provides methods for inducing an immune response in a subject by administering the disclosed compositions. The methods may be applied to any subject in need thereof, including humans and non-human mammals. The subject may be diagnosed with, suspected of having, or at risk of developing cancer.

The cancers treatable by the present compositions include, without limitation, head and neck squamous cell carcinoma, melanoma, lung adenocarcinoma, breast carcinoma, ovarian carcinoma, colorectal carcinoma, pancreatic carcinoma, prostate carcinoma, and bladder carcinoma. In some embodiments, the cancer expresses ORF1p at levels at least about 2-fold higher than adjacent normal tissue (H-score).

The composition may be administered by any route suitable for nucleic acid delivery, including intramuscular, subcutaneous, intravenous, intradermal, or intratumoral injection. Intramuscular administration into the deltoid or quadriceps is preferred for systemic immune activation. Doses may range from about 0.1 μg to about 500 μg of mRNA per administration, or about 1 μg/kg to about 100 μg/kg body weight. In some embodiments, two or more doses are administered in a prime-boost regimen separated by intervals of about 1 to 6 weeks.

In one embodiment, the composition is administered as a standalone vaccine to induce protective or therapeutic immunity. In another embodiment, the vaccine is co-administered with chemotherapeutic agents such as cisplatin, paclitaxel, or doxorubicin, or with radiotherapy, to enhance antigen release from tumor cells and promote immune priming.

The nanoparticles may be engineered for enhanced biodistribution or tumor targeting. For instance, particles may be coated with antibodies against EGFR, HER2, or PD-L1 for delivery to tumor cells, or with mannose residues for preferential uptake by dendritic cells in lymphoid tissues.

The compositions may also be employed as prophylactic vaccines in high-risk populations with genetic or environmental predispositions to cancer, or as therapeutic vaccines for patients with existing tumors. In therapeutic contexts, the vaccine may be administered alongside immune checkpoint inhibitors or cytokine therapies to potentiate antitumor immunity.

In certain embodiments, the composition elicits immune responses against multiple tumor types simultaneously due to the over-expression of ORF1p across diverse cancers. Thus, a single ORF1p mRNA-LNP formulation can serve as a pan-cancer immunotherapeutic platform.

### Formulation and Testing

The compositions may be prepared by microfluidic mixing of lipid and mRNA solutions, yielding self-assembling nanoparticles. The aqueous phase may contain the mRNA in citrate buffer (pH 4.0), while the organic phase contains lipids dissolved in ethanol. Mixing ratios of about 1:1 to about 5:1 (aqueous: organic) may be employed. The resulting nanoparticles are dialyzed or diafiltered into phosphate-buffered saline or other physiological buffers.

The formulations may be characterized by particle size, zeta potential, encapsulation efficiency, and mRNA integrity. Encapsulation efficiencies of at least about 80%, preferably about 90% or greater, are achieved. Particle sizes of about 60-120 nm ensure optimal lymphatic drainage and uptake by antigen-presenting cells.

Immunogenicity may be assessed in vitro using human or murine dendritic cell cultures, and in vivo using murine models. Flow cytometry, ELISpot, and cytokine assays may quantify immune responses. Successful vaccination results in expansion of ORF1p-specific CD8⁺ T cells, secretion of IFN-γ and TNF-α, and inhibition of tumor growth.

Efficacy may be tested in syngeneic tumor models expressing ORF1p, such as B16 melanoma or TC-1 carcinoma engineered for ORF1p overexpression. Humanized mouse models reconstituted with human immune cells may be used to evaluate human-specific responses.

Safety assessments may include histopathological evaluation, cytokine profiling, and serum biochemistry to confirm tolerability. The formulations are expected to be well tolerated, as LNP-based mRNA vaccines are non-replicating and transiently expressed.

### Additional Embodiments

In some embodiments, the mRNA encodes fusion proteins comprising ORF1p and immunostimulatory sequences, such as helper epitopes (PADRE, pan-DR) or trafficking signals (e.g., ubiquitin or lysosomal-targeting peptides) to enhance antigen presentation.

In other embodiments, multiple mRNA species encoding different tumor antigens, such as ORF1p and neoantigens specific to a patient's tumor, are co-formulated in a single nanoparticle preparation.

The invention also contemplates that nanoparticles may contain immune-modulatory RNA sequences, such as short double-stranded RNAs, to stimulate innate immunity.

The compositions may further include antioxidants (ascorbic acid, tocopherol), surfactants (Tween 20, Poloxamer 188), or tonicity agents (NaCl, sucrose) to improve stability and compatibility.

In summary, the present invention thus provides compositions and methods for eliciting potent, antigen-specific immune responses against cancers expressing LINE-1 ORF1p. The mRNA-LNP platform described herein is adaptable to a broad range of tumor types, delivery routes, and combination therapies. By targeting a tumor-specific antigen expressed across multiple malignancies, this technology offers a safe, versatile, and scalable strategy for cancer immunotherapy.

### Examples

The following examples show that an mRNA vaccine targeting LINE-1 ORF1 protein (ORF1p) is immunogenic in a syngeneic mouse model. In Example 1, an isogenic ORF1p-expressing mouse HNSCC cell line is created. In Example 2, an mRNA vaccine that synthesizes LINE-1 ORF1p antigen upon cellular uptake is tested. The ORF1p+ tumor cell line from Example 1 and ORF1pen coding mRNA from Example 2 are critical reagents for Example 3, where the safety and efficacy of the mRNA vaccine in a syngeneic mouse HNSCC model against ORF1p+ tumor cells is evaluated. These examples elucidate a new path for cancer immunotherapy.

These examples are innovative in both concept and its implementation. First, this is the first of its kind in developing a LINE-1 ORF1p-based therapeutic cancer vaccine. Current studies in the field focus on LINE-1's role in mutating the cancer cell genome and/or the role of its nucleic acid intermediates, such as the cytoplasmic complementary DNA as a trigger for sterile inflammation (Burns 2017; Grundy et al. 2022). Second, as compared with other TAAs, LINE-1 ORF1p has several unique properties: (a) Unlike neoantigens arising from mutated genes, LINE-1 ORF1p is encoded by the “normal” form. Thus, the vaccine requires no personalization, avoids the emergence of resistance due to target mutations, and can be a one-pill-fits-all solution. (b) Nearly half of human cancers show abnormal expression of LINE-1 ORF1p (Rodic et al. 2014). As such, the ORF1p-based cancer vaccine can benefit a significant fraction of the cancer patients. (c) LINE-1 ORF1p is neither expressed in normal somatic cells nor required for normal cellular functions. The likelihood of severe adverse reactions such as autoimmune pathology is low. Third, the prototypic LINE-1 ORF1p cancer vaccine takes advantage of the cutting-edge mRNA vaccine platform, which has shown remarkable success in SARS-COV-2 vaccines as well as great promise for cancer vaccines.

### Example 1—In Vitro Characterization of LINE-1 ORF1p mRNA

Disclosed herein is the rationale and design strategy for developing a cancer-targeted mRNA vaccine encoding the LINE-1 ORF1p protein. LINE-1 retrotransposons are reactivated in multiple cancer types and serve as promising tumor-associated antigens due to their abnormal expression in malignant cells.

LINE-1 ORF1p is one of the two proteins encoded by a full-length LINE-1 mRNA. The detection of ORF1p expression in cancer dates back to the early 90s and was first reported in human testicular cancer and breast cancer samples (Bratthauer and Fanning 1992; Asch et al. 1996; Su et al. 2007; Harris et al. 2010; Chen et al. 2012). Later, a comprehensive survey of 25 cancer histotypes identified abnormal expression of ORF1p in 47% of the samples (Rodic et al. 2014). Importantly, LINE-1 ORF1p signal is restricted to tumor cells but not in adjacent histologically normal cells. Hence, an ORF1p-based mRNA vaccine can benefit a significant number of cancer patients. An mRNA vaccine targeting LINE-1 ORF1p is developed and initial characterization is performed in cultured cells using a transient overexpression approach.

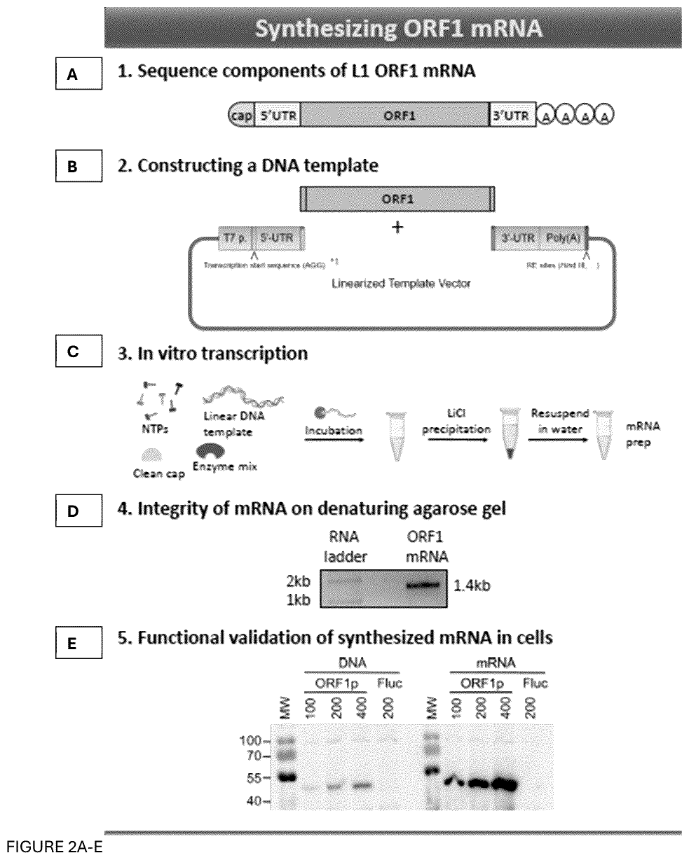

To create DNA templates for in vitro transcription (IVT), two DNA templates for in vitro transcription of mouse LINE-1 ORF1p mRNA are created: one is based on the endogenous mouse LINE-1 sequence and the other is the codon-optimized version (Naas et al. 1998; Han and Boeke 2004) (FIG. 2A). The Takara IVTpro mRNA Synthesis System is used to construct the plasmid template and to generate high-yield mRNA (FIG. 2B). Briefly, ORF1p coding sequence is PCR amplified from an existing donor plasmid containing a full-length LINE-1. The resultant PCR amplicon contains the ORF1p coding sequence that is flanked by two 15-base homologous arms designed for In-Fusion cloning with the linearized vector backbone. The final plasmid consists of T7 promoter, transcription start sequence AG (modified from GG to be compatible with cotranscriptional capping using CleanCap AG reagent), 5′UTR (untranslated region), ORF1p coding sequence, 3′UTR, and 105-base poly (A) sequence. A Kozak consensus sequence (GCCACCATGG, where the underlined ATG denotes the start codon) is maintained at the junction between 5′UTR and ORF1p coding sequence. At the junction between ORF1p coding sequence and 3′UTR are two consecutive stop codons (TAATGA), which ensures efficient translation termination. Downstream of the poly(A) sequence is a cluster of restriction sites that can be used to linearize the template plasmid (FIG. 2B).

To synthesize ORF1p mRNA by IVT. The template plasmid pRN01 is linearized and used for IVT with unmodified nucleoside triphosphates (NTPs) (FIG. 2C). For experiments requiring enhanced mRNA stability and reduced innate immune recognition, mRNA was synthesized using modified-N1-methylpseudouridine-5′-Triphosphate (YUTP, also known as pseudouridine) (Trilink, Cat. No. N-1081) instead of using unmodified UTP. CleanCap Reagent AG (3′OMe) (TriLink Biotechnologies) are added, enabling co-transcriptional capping of synthesized mRNA and forming a cap 1 structure consisting of 5′ N7-methyl-3′-O-methylguanosine linked via a 5′-5′ triphosphate bridge to 2′-Omethylguanosine. The 2′-O-methylation at the +1 nucleotide is a signature of self RNA (Daffis et al. 2010). The additional 3′-O-methyl modification of the m7G cap ensures efficient transcriptional initiation only in the desired forward orientation (Peng et al. 2002). The IVT reaction product is treated with DNase I to remove the DNA template. The synthesized mRNA is recovered by lithium chloride precipitation. The length and purity of the RNA are confirmed by agarose gel electrophoresis (FIG. 2D). Firefly luciferase (Fluc) mRNA is synthesized and serve as a control. A single 20 μl reaction can yield up to 200 μg of mRNA.

To validate ORF1p mRNA function by transient transfection. The function and integrity of synthesized mRNA is confirmed in mouse cells. In a pilot experiment, a mouse melanoma cell line, B16F10 (ATCC #CRL-6475), was transfected with codon-optimized ORF1p mRNA or Fluc mRNA in a 24-well plate. To quantify ORF1p expression by Western blot, cells were harvested 48 hours post transfection and cell lysates were probed by a monoclonal antibody specific for mouse ORF1p (FIG. 2E). While ORF1p is undetectable in B16F10 cells transfected with the control Fluc mRNA, a dose-dependent increase of ORF1p signal was detected in cells transfected with ORF1p mRNA. Importantly, higher levels of ORF1p was expressed from mRNA than from identical amount of the template DNA plasmid (FIG. 2E).

### Example 2—Formulating and In Vitro Testing of ORF1p mRNA Vaccine

Disclosed herein is the design and formulation of a preclinical mRNA vaccine targeting the LINE-1 ORF1p protein, a tumor-associated antigen derived from LINE-1 retrotransposons. The vaccine was developed using in vitro transcription (IVT) and lipid nanoparticle (LNP) encapsulation techniques. The mRNA construct was optimized with untranslated regions (UTRs), a synthetic poly (A) tail, and co-transcriptional capping to enhance stability and translation efficiency. Two versions of the mRNA, one with unmodified uridine and another with NI-methylpseudouridine, were synthesized and compared. The IVT reactions consistently produced full-length transcripts (˜1.4 kb), and RNA integrity was confirmed via gel electrophoresis. LNPs were formulated using a microfluidic mixer and a defined lipid composition, resulting in nanoparticles within the optimal size range (160-220 nm) for lymphatic drainage and dendritic cell uptake. Dynamic light scattering (DLS) analysis showed low polydispersity index (PDI), indicating uniform particle distribution. Zeta potential measurements revealed mildly negative surface charges, supporting colloidal stability. Encapsulation efficiency was assessed using the RiboGreen assay, with values ranging from 74% to 92% across formulations. Post-formulation RNA integrity remained intact, confirming the protective role of LNPs., this study establishes a reliable workflow for synthesizing and formulating mRNA-LNP vaccines and highlights the potential of LINE-1 ORF1p as a cancer immunotherapy target.

The mRNA vaccine platform has gained momentum due to the overwhelming success of SARS-COV2 vaccines and is now being used in multiple cancer vaccine trials (Lorentzen et al. 2022; Raimondo et al. 2023). In a recent phase I clinical trial, an individualized neoantigen mRNA vaccine produced by BioNTech elicited strong T cell response and promoted survival in eight out of the 16 pancreatic cancer patients (Rojas et al. 2023). Thus, a similar formulation is prepared for ORF1p mRNA vaccine to evaluate its safety and immunogenicity.

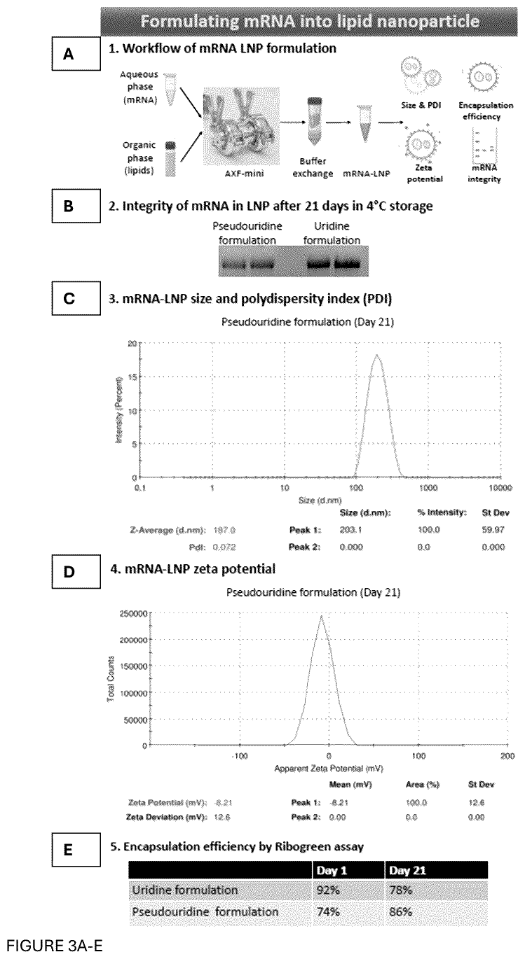

To prepare mRNA lipid nanoparticle (LNP) complex (FIG. 3A). To formulate mRNA for in vivo delivery, the ORF1p mRNA is encapsulated using LNP-102 Kit (Cayman Chemical). The kit includes four different lipids: (1) SM-102: an ionizable cationic lipid that encapsulates mRNA during particle formation and facilitates membrane fusion during internalization, (2) 1,2-DSPC: a phospholipid that forms the lipid bilayer, (3) cholesterol: a natural component of cellular membrane that modulates membrane fluidity and promotes LNP stability, and (4) DMGPEG2000: a polyethylene glycol lipid that controls particle size and prevents aggregation during storage. The same four lipids were used by formulating Moderna's SARS-COV-2 mRNA-1273 vaccine (Corbett et al. 2020; Schoenmaker et al. 2021). The four lipids are used at a ratio of 50:10:38.5:1.5 and mixed with mRNA at a ratio of 2.5:1 (lipids: mRNA).

Following the emulsification from AXF mini, the mRNA-loaded lipid nanoparticles formulation in the acidic buffer system was subjected to buffer exchange into phosphate-buffered saline (PBS, pH 7.4) to ensure physiological compatibility for in vivo use. The procedure was done using Amicon® Ultra centrifugal filter units, 100 kDa molecular weight cut-off (Merck Millipore). The final solution is filter sterilized and frozen for downstream analyses.

To extract mRNA from LNPs for integrity assessment, we used the RNeasy Plus Mini Kit (Qiagen, Cat. No. 74134) following the manufacturer's protocol. The integrity of encapsulated mRNA is tested by agarose gel electrophoresis (FIG. 3B).

The size and zeta potential of the formulation were measured using dynamic light scattering (DLS) (Malvern Zeta Sizer). The formulation was diluted 1:10 in PBS to measure the size. 10 μl of the formulation and 90 μl of PBS were added in a cuvette (ZEN2112) and the size was measured (FIG. 3C). Zeta potential (ζ-potential) is a critical indicator of nanoparticle surface charge and stability. It influences colloidal dispersion, aggregation tendencies, and interactions with cell membranes, particularly antigen-presenting cells such as dendritic cells. To measure the zeta potential, the formulation was diluted 1:20 in PBS, by taking 20 ul of formulation in 980 ul of PBS in a disposable cuvette (DTS 1020), and was added to the instrument to record the reading (FIG. 3D).

The encapsulation efficiency is determined by Quant-iT™ RiboGreen RNA assay kit (Thermo Fisher Scientific, Cat. No. R11490) (FIG. 3E).

### Example 3—In Vivo Testing of ORF1p mRNA Vaccine

Disclosed herein is the preclinical evaluation of the immunogenicity of a LINE-1 ORF1p mRNA vaccine in C57BL/6 mice. Following a prime-boost regimen, splenocytes were analyzed for cytokine production by the activated T cells. Notably, ORF1p mRNA group demonstrated significant increases in IFN-γ and TNF-α production in both CD4 and CD8⁺ T cells upon PMA ionomycin stimulation, suggesting the mRNA vaccine led to the expansion of functional memory T cells. Overall the data are consistent with enhanced functional T cell responses in the ORF1p mRNA group. These findings at the pre-clinical level provide a foundation for future development of targeting LINE-1 ORF1p in cancer immunotherapy.

To evaluate the safety and immunogenicity of mRNA LNP, two groups of 6-week-old C57BL/6J mice are immunized intramuscularly twice with 1× phosphate buffered saline (PBS; the control group), or 0.5 μg of mRNA-LNP, respectively. For safety, mice are monitored for appearance and behavior daily and weighed three times a week. For immunogenicity, cellular immune responses are examined three weeks post boost.

Single cell suspensions of splenocytes are analyzed for T cell activation by flow cytometry. Spleen tissues were processed by transferring them into petri dishes containing 5 mL DMEM. The spleens were dissociated mechanically using a syringe plunger to create a single-cell suspension. This suspension was passed through a 40 μm cell strainer into 50 mL Falcon tubes and centrifuged at 300×g for 10 minutes at 4° C. RBC lysis was performed using freshly prepared 1×RBC lysis buffer, incubated at room temperature for 5 minutes, and neutralized by adding 5 volumes of DMEM. After centrifugation, the supernatant was discarded, and cells were resuspended in 3 mL of DMEM for direct counting and staining or cryopreservation.

To detect intracellular cytokines in splenocytes, cells are first stimulated using a commercial Cell Activation Cocktail containing Phorbol myristate acetate (PMA), ionomycin, and Brefeldin A (BioLegend, Cat #423303) at a final concentration of 1× for 4 hours. Following stimulation, the cells were pelleted and subjected to viability staining using the Zombie UV Fixable Viability Dye (BioLegend, Cat #423108). To preserve cellular morphology and intracellular contents, cells were then fixed using 100 μL of Cyto-Fast Fixation Buffer (BioLegend, Cat #426803) and incubated for 20 minutes at room temperature in the dark. The fixation was followed by permeabilization using 1X Cyto-Fast Perm Wash buffer. Fc receptors and non-specific monocyte binding sites were blocked using a 50 μL blocking cocktail per sample, followed by antibody staining. The antibody panel includes CD3 (T cell marker), CD4 (T helper cells), CD8 (cytotoxic T cells), IFN-γ, and TNF-α. Samples were acquired on a BD FACS LSR Fortessa SORP (five-laser, 18-color) flow cytometer. The proportion of IFN-γ+ and TNF-α+CD4+ and CD8+ T cells is significantly increased in the vaccinated group, indicating robust immune response to the ORF1p mRNA vaccine.

### Example 4—Creating Syngeneic Mouse Tumor Models

To conduct preclinical testing of ORF1p mRNA vaccines, syngeneic mouse tumor models are required. To this end, a transgene encoding a codon optimized mouse ORF1p is integrated into two mouse separate mouse tumor cell lines: the B16F10 melanoma cell line (FIG. 7A) and the mEERL head and neck cancer cell line (FIG. 7B). Western blot demonstrates robust expression of ORF1p in selected cell clones.

### Conclusions:

LINE-1 ORF1 mRNA-LNP formulations have been produced with satisfying quality parameters (size, charge, encapsulation efficiency, and translatability in cells).

mRNA integrity is maintained after storage at 4° C. for three weeks.

Intramuscular injection of two doses of uridine or pseudouridine-containing ORF1 mRNA LNP is safe in wild-type C57BL/6 mice.

Assessment of immunogenicity is ongoing by using multicolor flow cytometry for surface and intracellular markers of immune cell populations. Antigen-specific T cell responses are measured by IFN-γ ELISpot assays.

Two syngeneic ORF1+ mouse cancer cell lines of C57BL/6 background (head/neck and melanoma) have been generated and are used in efficacy testing. The isogenic ORF1-clones serve as a control for the specificity of ORF1 mRNA LNP vaccine.

In parallel, human LINE-1 ORF1-based mRNA LNP are tested in humanized mouse models.

Combination with immune checkpoint inhibitors, chemotherapy, radiotherapy, and/or other adjuvant therapies can be used to enhance ORF1 mRNA LNP's effects.

### REFERENCES

Andries O, Mc Cafferty S, De Smedt S C, Weiss R, Sanders N N, Kitada T. 2015. N (1)-methylpseudouridineincorporated mRNA outperforms pseudouridine-incorporated mRNA by providing enhanced protein expression and reduced immunogenicity in mammalian cell lines and mice. J Control Release 217:337-344.
Ardeljan D, Taylor M S, Ting D T, Burns K H. 2017. The Human Long Interspersed Element-1 Retrotransposon: An Emerging Biomarker of Neoplasia. Clin Chem 63:816-822.
Asch H L, Eliacin E, Fanning T G, Connolly J L, Bratthauer G, Asch B B. 1996. Comparative expression of the LINE-1 p40 protein in human breast carcinomas and normal breast tissues. Oncol Res 8:239-247.
Bermejo A V, Nielsen K N, Holst P J. 2021. Comment on: Endogenous retroviruses expressed in human tumours cannot be used as targets for anti-tumour vaccines. Transl Oncol 14:101040.
Bratthauer G L, Fanning T G. 1992. Active LINE-1 retrotransposons in human testicular cancer. Oncogene 7:507-510.
Budania S, Sur D, Nangal J, Pilli S, Mukherjee K, Biswas M, Prasad R, Saxena S, Mandal P K. 2020. LINE-1 retrotransposon encoded ORF1p expression and promoter methylation in oral squamous cell carcinoma: a pilot study. Cancer Genet 244:21-29.
Burns K H. 2017. Transposable elements in cancer. Nat Rev Cancer 17:415-424.
Callejas-Valera J L, Vermeer D W, Lucido C T, Williamson C, Killian M, Vermeer P D, Spanos W C, Powell S F. 2022. Characterization of the Immune Response to PD-1 Blockade during Chemoradiotherapy for Head and Neck Squamous Cell Carcinoma. Cancers (Basel) 14.
Chen L, Dahlstrom J E, Chandra A, Board P, Rangasamy D. 2012. Prognostic value of LINE-1 retrotransposon expression and its subcellular localization in breast cancer. Breast Cancer Res Treat 136:129-142.
Corbett K S, Edwards D K, Leist S R, Abiona O M, Boyoglu-Barnum S, Gillespie R A, Himansu S, Schafer A, Ziwawo C T, DiPiazza A T et al. 2020. SARS-COV-2 mRNA vaccine design enabled by prototype pathogen preparedness. Nature 586:567-571.
Daffis S, Szretter K J, Schriewer J, Li J, Youn S, Errett J, Lin T Y, Schneller S, Zust R, Dong H et al. 2010. 2′-O methylation of the viral mRNA cap evades host restriction by IFIT family members. Nature 468:452-456.
Goodier J L, Ostertag E M, Du K, Kazazian H H, Jr. 2001. A novel active L1 retrotransposon subfamily in the mouse. Genome Res 11:1677-1685.
Grandi F C, Rosser J M, Newkirk S J, Yin J, Jiang X, Xing Z, Whitmore L, Bashir S, Ivics Z, Izsvak Z et al. 2015.
Retrotransposition creates sloping shores: a graded influence of hypomethylated CpG islands on flanking CpG sites. Genome Res 25:1135-1146.
Grundy E E, Diab N, Chiappinelli K B. 2022. Transposable element regulation and expression in cancer. The FEBS journal 289:1160-1179.
Han J S, Boeke J D. 2004. A highly active synthetic mammalian retrotransposon. Nature 429:314-318.
Harris C R, Normart R, Yang Q, Stevenson E, Haffty B G, Ganesan S, Cordon-Cardo C, Levine A J, Tang L H.
2010. Association of Nuclear Localization of a Long Interspersed Nuclear Element-1 Protein in Breast Tumors with Poor Prognostic Outcomes. Genes Cancer 1:115-124.
Hoover A C, Spanos W C, Harris G F, Anderson M E, Klingelhutz A J, Lee J H. 2007. The role of human papillomavirus 16 E6 in anchorage-independent and invasive growth of mouse tonsil epithelium. Arch Otolaryngol Head Neck Surg 133:495-502.
Hudecek M, Izsvak Z, Johnen S, Renner M, Thumann G, Ivics Z. 2017. Going non-viral: the Sleeping Beauty transposon system breaks on through to the clinical side. Crit Rev Biochem Mol Biol 52:355-380.
Ivics Z, Hackett P B, Plasterk R H, Izsvak Z. 1997. Molecular reconstruction of Sleeping Beauty, a Tcl-like transposon from fish, and its transposition in human cells. Cell 91:501-510.
Kariko K, Muramatsu H, Welsh F A, Ludwig J, Kato H, Akira S, Weissman D. 2008. Incorporation of pseudouridine into mRNA yields superior nonimmunogenic vector with increased translational capacity and biological stability. Mol Ther 16:1833-1840.
Ke N, Albers A, Claassen G, Yu D H, Chatterton J E, Hu X, Meyhack B, Wong-Staal F, Li Q X. 2004. One-week 96-well soft agar growth assay for cancer target validation. Biotechniques 36:826-828, 830, 832-823.
Laczko D, Hogan M J, Toulmin S A, Hicks P, Lederer K, Gaudette B T, Castano D, Amanat F, Muramatsu H, Oguin T H, 3rd et al. 2020. A Single Immunization with Nucleoside-Modified mRNA Vaccines Elicits Strong Cellular and Humoral Immune Responses against SARS-COV-2 in Mice. Immunity 53:724-732 e727.
Lorentzen C L, Haanen J B, Met O, Svane I M. 2022. Clinical advances and ongoing trials on mRNA vaccines for cancer treatment. Lancet Oncol 23: e450-e458.
Martin S L. 1991. Ribonucleoprotein particles with LINE-1 RNA in mouse embryonal carcinoma cells. Mol Cell Biol 11:4804-4807.
Martin S L, Branciforte D. 1993. Synchronous expression of LINE-1 RNA and protein in mouse embryonal carcinoma cells. Mol Cell Biol 13:5383-5392.
Mates L, Chuah M K, Belay E, Jerchow B, Manoj N, Acosta-Sanchez A, Grzela D P, Schmitt A, Becker K, Matrai J et al. 2009. Molecular evolution of a novel hyperactive Sleeping Beauty transposase enables robust stable gene transfer in vertebrates. Nat Genet 41:753-761.
Naas T P, DeBerardinis R J, Moran J V, Ostertag E M, Kingsmore S F, Seldin M F, Hayashizaki Y, Martin S L, Kazazian H H. 1998. An actively retrotransposing, novel subfamily of mouse L1 elements. EMBO J 17:590-597.
Nelson J, Sorensen E W, Mintri S, Rabideau A E, Zheng W, Besin G, Khatwani N, Su S V, Miracco E J, Issa W J et al. 2020. Impact of mRNA chemistry and manufacturing process on innate immune activation. Sci Adv 6: eaaz6893.
Niwa H, Yamamura K, Miyazaki J. 1991. Efficient selection for high-expression transfectants with a novel eukaryotic vector. Gene 108:193-199.
Peng Z H, Sharma V, Singleton S F, Gershon P D. 2002. Synthesis and application of a chain-terminating dinucleotide mRNA cap analog. Org Lett 4:161-164.
Raimondo T M, Reed K, Shi D, Langer R, Anderson D G. 2023. Delivering the next generation of cancer immunotherapies with RNA. Cell 186:1535-1540.
Rastogi I, Muralidhar A, McNeel D G. 2023. Vaccines as treatments for prostate cancer. Nat Rev Urol doi: 10.1038/s41585-023-00739-w: 1-16.
Rodic N, Sharma R, Zampella J, Dai L, Taylor M S, Hruban R H, Iacobuzio-Donahue C A, Maitra A, Torbenson M S, Goggins M et al. 2014. Long Interspersed Element-1 Protein Expression Is a Hallmark of Many Human Cancers. Am J Pathol doi: S0002-9440 (14) 00077-7 [pii] 10.1016/j.ajpath.2014.01.007.
Rojas L A, Sethna Z, Soares K C, Olcese C, Pang N, Patterson E, Lihm J, Ceglia N, Guasp P, Chu A et al. 2023. Personalized RNA neoantigen vaccines stimulate T cells in pancreatic cancer. Nature doi: 10.1038/s41586-023-06063-y.
Saha P S, An W. 2020. Recently Mobilised Transposons in the Human Genome. In eLS, doi: 10.1002/9780470015902.a0020837, pp. 1-10. John Wiley & Sons, Ltd (Ed.).
Saxena M, van der Burg S H, Melief C J M, Bhardwaj N. 2021. Therapeutic cancer vaccines. Nat Rev Cancer 21:360-378.
Schichman S A, Severynse D M, Edgell M H, Hutchison C A, 3rd. 1992. Strand-specific LINE-1 transcription in mouse F9 cells originates from the youngest phylogenetic subgroup of LINE-1 elements. J Mol Biol 224:559-574.
Schoenmaker L, Witzigmann D, Kulkarni J A, Verbeke R, Kersten G, Jiskoot W, Crommelin DJA. 2021. mRNAlipid nanoparticle COVID-19 vaccines: Structure and stability. Int J Pharm 601:120586.
Siegel R L, Miller K D, Wagle N S, Jemal A. 2023. Cancer statistics, 2023. C A Cancer J Clin 73:17-48.
Sookdeo A, Hepp C M, McClure M A, Boissinot S. 2013. Revisiting the evolution of mouse LINE-1 in the genomic era. Mob DNA 4:3.
Su Y, Davies S, Davis M, Lu H, Giller R, Krailo M, Cai Q, Robison L, Shu X O. 2007. Expression of LINE-1 p40 protein in pediatric malignant germ cell tumors and its association with clinicopathological parameters: a report from the Children's Oncology Group. Cancer Lett 247:204-212.
Taddei M L, Giannoni E, Fiaschi T, Chiarugi P. 2012. Anoikis: an emerging hallmark in health and diseases. J Pathol 226:380-393.
Tomayko M M, Reynolds C P. 1989. Determination of subcutaneous tumor size in athymic (nude) mice. Cancer Chemother Pharmacol 24:148-154.
Upadhaya S, Hubbard-Lucey V M, Yu J X. 2020. Immuno-oncology drug development forges on despite COVID-19. Nat Rev Drug Discov 19:751-752.
Verbeke R, Hogan M J, Lore K, Pardi N. 2022. Innate immune mechanisms of mRNA vaccines. Immunity 55:1993-2005.
Vergara Bermejo A, Ragonnaud E, Daradoumis J, Holst P. 2020. Cancer Associated Endogenous Retroviruses: Ideal Immune Targets for Adenovirus-Based Immunotherapy. Int J Mol Sci 21.
Vermeer D W, Coppock J D, Zeng E, Lee K M, Spanos W C, Onken M D, Uppaluri R, Lee J H, Vermeer P D. 2016.
Metastatic model of HPV+ oropharyngeal squamous cell carcinoma demonstrates heterogeneity in tumor metastasis. Oncotarget 7:24194-24207.
Vermeer D W, Spanos W C, Vermeer P D, Bruns A M, Lee K M, Lee J H. 2013. Radiation-induced loss of cell surface CD47 enhances immune-mediated clearance of human papillomavirus-positive cancer. Int J Cancer 133:120-129.
Vogel A B, Kanevsky I, Che Y, Swanson K A, Muik A, Vormehr M, Kranz L M, Walzer K C, Hein S, Güler A et al. 2020. A prefusion SARS-COV-2 spike RNA vaccine is highly immunogenic and prevents lung infection in non-human primates. bioRxiv doi: 10.1101/2020.09.08.280818:2020. 2009.2008.280818.
Waterston R H Lindblad-Toh K Birney E Rogers J Abril J F Agarwal P Agarwala R Ainscough R Alexandersson M An P et al. 2002. Initial sequencing and comparative analysis of the mouse genome. Nature 420:520-562.
Williams R, Lee D W, Elzey B D, Anderson M E, Hostager B S, Lee J H. 2009. Preclinical models of HPV+ and HPV-HNSCC in mice: an immune clearance of HPV+HNSCC. Head Neck 31:911-918.
Xie Y, Mates L, Ivics Z, Izsvak Z, Martin S L, An W. 2013. Cell division promotes efficient retrotransposition in a stable L1 reporter cell line. Mob DNA 4:10.

1. A composition comprising a nucleic acid encoding long interspersed elements type 1 open reading frame 1 protein (LINE-1 ORF1p) or a functional variant thereof, and a nanoparticle.

2. The composition of claim 1, wherein the nucleic acid is mRNA.

3. The composition of claim 2, wherein the mRNA comprises at least one of these characteristics:
(a) codon-optimized for mammalian expression;
(b) modified to include N1-methylpseudouridine or unmodified uridine;
(c) flanked by 5′ and 3′ untranslated regions (UTRs); and
(d) capped with a co-transcriptional cap structure.

4. The composition of claim 1, wherein said composition is configured for delivery to antigen-presenting cells and can induce an immune response against ORF1p-expressing tumor cells.

5. The composition of claim 1, wherein the nanoparticle is a lipid nanoparticle (LNP) comprising ionizable lipids, cholesterol, phospholipids, and/or polyethylene glycol (PEG)-lipids.

6. The composition of claim 1, wherein the nanoparticle is a polymeric nanoparticle.

7. The composition of claim 1, wherein the nanoparticle includes targeting ligands.

8. The composition of claim 1, wherein the nanoparticle further comprises adjuvants.

9. The composition of claim 1, wherein the nanoparticle includes DSPC.

10. The composition of claim 1, further comprising cryoprotectants for storage stability.

11. The composition of claim 1, further comprising untranslated regions for regulated translation.

12. A method of eliciting an immune response in a subject, comprising delivering a composition to the subject, wherein the composition comprises a nucleic acid encoding long interspersed elements type 1 open reading frame 1 protein (LINE-1 ORF1p) or a functional variant thereof, encapsulated within a nanoparticle.

13. The method of claim 12, wherein the subject has a tumor characterized by the overexpression of LINE-1 ORF1 protein, and wherein administration induces a cytotoxic T lymphocyte (CTL) response specific to LINE-1 ORF1p-expressing tumor cells.

14. The method of claim 12, wherein the composition is formulated as a vaccine.

15. The method of claim 12, wherein the delivery is targeted directly to tumor cells.

16. The method of claim 12, wherein the delivery utilizes ligand-conjugated nanoparticles.

17. The method of claim 12, wherein the composition is co-administered with another anti-cancer agent and/or with adjuvants to enhance the immune response.

18. The method of claim 12, wherein the nanoparticles are engineered for enhanced endosomal escape.

19. The method of claim 12, wherein the composition promotes an immune response against multiple tumor types.

20. The method of claim 12, wherein the nanoparticle is modified with targeting ligands specific to cancer cell surface markers.


SEQUENCE LISTING (3 sequences)

SEQ ID NO: 1 - DNA, 27 bp, synthetic construct
  misc_feature 1..27: note = Description of sequence: primer
  source 1..27: mol_type = other DNA
agagaacccgccaccatggccaagggc

SEQ ID NO: 2 - DNA, 37 bp, synthetic construct
  misc_feature 1..37: note = Description of sequence: primer
  source 1..37: mol_type = other DNA
cgaggctccagctcattagcggcgggtcttctccagg

SEQ ID NO: 3 - DNA, 17 bp, synthetic construct
  misc_feature 1..17: note = Description of sequence: primer
  source 1..17: mol_type = other DNA
taatacgactcactata